(19) United States
(12) Patent Application Publication
(10) Pub. No.: US 2005/0186258 A1
(43) Pub. Date: Aug. 25, 2005

(54) Antimicrobial medical gloves

(76) Inventors: Shiping Wang, Libertyville, IL (US); Yun-Siung Tony Yeh, Libertyville, IL (US); Danny W. Penny, Third Lake, IL (US)

Correspondence Address: ALLEGIANCE CORPORATION;Attn: Kim Luna, MPKB-1A, 1430 Waukegan Road, McGaw Park, IL, 60085-6787, US

(21) Appl. No.: 10/783,362

(22) Filed: Feb. 20, 2004

(51) Int. Cl. A61K 9/70

(52) U.S. Cl. 424/443

(57) ABSTRACT

The invention disclosed herein relates to elastomeric articles such as medical or industrial gloves coated by antimicrobial compositions and protected by water-resistant packaging. Antimicrobial gloves are useful in methods for reducing nosocomial infection by Gram positive bacteria, Gram negative bacteria, fungi, and viruses. Packaged gloves maintain quick-kill activity against microbes, even after extended storage. In a preferred embodiment, the present invention provides a method of packaging which protects the antimicrobial activity of a glove during storage and transportation by shielding the glove from warm and/or humid environments. Alternatively, the present invention provides a system comprising an antimicrobial glove and packaging as a means for maintaining a low level of humidity in the vicinity of the glove.

### BACKGROUND OF THE INVENTION

The present invention relates to antimicrobial gloves and a means for packaging them which allows the gloves to maintain antimicrobial activity during storage. The gloves are particularly, but not exclusively, useful for medical applications, for example as both exam and surgical gloves. The medical gloves of the invention exhibit “quick-kill” activity against a broad spectrum of microorganisms and maintain their antimicrobial activity after being stored and transported under warm and humid environments. The invention may alternatively have application in other skin protection elements including but not limited to food-contact gloves, dental gloves, industrial gloves, biologically protective gloves, and also elastomeric articles such as medical devices, catheters, protective covers, and tubes.

Gloves have become an everyday part of clinical practice for healthcare workers and function as an element of personal protective equipment. Nosocomial (i.e. within and among hospital patients and staff) transmission of microorganisms can be reduced by compliance with handwashing and glove isolation procedures. However, investigations have found that for a variety of reasons the use of regular medical gloves alone does not provide sufficient protection against nosocomial transmission of microorganisms. For example, in many hospitals, healthcare workers do not don or change gloves as often as they should. A staff member's soiled glove can touch other areas such as a different resident, themselves, or an area surface, potentially resulting in patient-to-patient transmission of microorganisms. All workers do not thoroughly wash their hands before and after glove removal. Inappropriate management of contaminated gloves can result in cross-infection of hospital staff and patients.

The healthcare implications of nosocomial infection are large. According to the Centers for Disease Control and Prevention (CDC), 5-10% of patients contract infections while in hospitals, a figure that represents between 1.75 and 3.5 million Americans each year. One analysis found an estimated 103,000 deaths linked to hospital infection in 2000.

Efforts have been made to improve the quality of medical gloves in order to reduce nosocomial infections, but there has not been a satisfactory solution of the problems associated with such medical gloves. For example, U.S. Pat. No. 5,487,896 discloses a powdered antimicrobial glove coated by a chlorhexidine-cationic surfactant-starch lubricant slurry on the user's-side surface for rapid release of the anti-infective agent chlorhexidine. However, the efficacy of the antimicrobial glove described therein is limited to Staphylococcus aureus. U.S. Pat. No. 5,089,205 discloses a process for making powdered antimicrobial gloves by coating chlorhexidine gluconate (CHG) or chlorhexidine diacetate with polyester-urethane and p-chloro-m-xylenol (PCMX) on the user's-side surface. Unfortunately, the growth inhibition of Staphylococcus aureus stated in the disclosure is insufficient to quickly kill bacteria on contact. U.S. Pat. No. 6,488,948 discloses an anti-bacterial glove coating, containing CHG or benzalkonium chloride (BKC), applied on the user's-side surface of uncured gloves followed by oven-curing. The antibacterial activity of the glove is limited because the coating composition is applied to the inside surface of the glove rather than the outside surface of the glove. U.S. Pat. No. 4,853,978 discloses an antimicrobial surgical glove made with a water-based coating containing a polyurethane dispersion, CHG as an antimicrobial agent, starch powder and a cationic surfactant. The disclosure claims that the release of antimicrobial agent is slow, thereby limiting its efficacy.

In addition, the disclosures discussed above do not mention other corollary factors impacting the efficacy of antimicrobial gloves, namely the effect of storage and transportation on antimicrobial activities. The inventors of the instant invention have found that warm and/or humid environments, which occur during storage and transportation, accelerate the diffusion of the antimicrobial agent coated on the surface into the glove substrate, thereby reducing the surface concentration of the antimicrobial agent to a level that is ineffective in killing microorganisms. Therefore, in addition to the need for effective antimicrobial gloves, there is a need for medical gloves which provide effective antimicrobial activity against hospital microorganisms to improve the protection of patient and staff from the risk of infection even after storage of the gloves in warm and/or humid environments.

One objective of the invention is to develop a medical glove with additional protection for both patient and staff without sacrificing other glove properties. Another objective of the invention is to develop an antimicrobial glove which provides efficacy against a broad spectrum of microorganisms in minutes. An additional objective of the invention is to develop packaging which protects antimicrobial gloves from a loss of antimicrobial activity due to warm and/or humid environments.

### SUMMARY OF THE INVENTION

Water-soluble antimicrobial agents in a coating formulation and a means of packaging to protect the antimicrobial activity of a coated elastomeric article against warm and/or humid environments can be used for making antimicrobial articles which overcome the drawbacks discussed above. According to the present invention, there is provided
A) an elastomeric article
B) a water-based antimicrobial solution comprising:
   1. an antimicrobial mixture comprising
      i) at least one water-soluble chlorhexidine salt, preferably chlorhexidine gluconate (CHG) at about 0.01% to about 4% by weight; and
      ii) at least one water-soluble quaternary ammonium halide, preferably benzalkonium chloride (BKC), benzethonium chloride (BZT), and/or cetyl pyridinium chloride (CPC) at about 0.5% to about 4% by weight; and
   2. an aqueous carrier with or without a solvent;
   and optionally comprising one or more of the following:
   3. a wetting agent, preferably a polyether-modified dimethylpolysiloxane such as BYK-348 at about 0.01% to about 0.5% by weight, which improves coating coverage;
   4. an anti-foaming agent, preferably a self-emulsifiable acetylenic diol such as Surfynol TG at about 0.01% to about 0.3% by weight, which reduces coating defects due to dynamic surface tension reduction;
   5. a buffer or pH adjusting agent, preferably citric acid at about 0.05% by weight or as necessary to buffer or adjust the pH;
   6. a chelating agent, such as salts of ethylenediamine tetraacetic acid, preferably disodium ethylenediamine tetraacetate, Na₂ EDTA at about 0.1% to about 0.5% by weight; and
   7. an anti-tackifying agent; and
C) a package to protect the antimicrobial activity of the elastomeric article against warm and/or humid environments.

In a preferred embodiment, the present invention provides a method of packaging which protects the antimicrobial activity of a glove during storage and transportation by shielding the glove from warm and/or humid environments, comprising: placing the gloves within a means for reducing the relative humidity in the vicinity of the glove to less than the ambient relative humidity, preferably comprising a moisture-resistant barrier or metal foil pouch containing a desiccant.

In a particularly preferred embodiment, the present invention provides a system comprising an antimicrobial glove and packaging to protect the antimicrobial activity of the glove during storage and transportation. The packaging comprises a means for maintaining a low level of humidity in the vicinity of the glove.

### DETAILED DESCRIPTION OF THE INVENTION

Gram negative bacteria contain an outer cytoplasmic membrane consisting of lipopolysaccharide molecules that surround the cell wall serving as a selective permeability barrier between the cytoplasm and the cell environment. Gram positive bacteria do not have an outer membrane, only the inner cytoplasmic membrane consisting of phospholipids and protein. Both Gram positive and Gram negative bacteria are found in a hospital environment.

The phrase “broad spectrum” with respect to microorganisms includes without limitation Gram positive bacteria such as Staphylococcus aureus and Enterococcus faecalis, and Gram negative bacteria such as Escherichia coli and Pseudomonas aeroginosa, yeasts such as Candida albicans, and clinical isolates such as methicillin-resistant Staphylococcus aureus (MRSA) and vancomycin-resistant Enterococcus faecium (VRE).

Chlorhexidine salts are chlorine-containing cationic organic biguanides with wide spectrum activity against bacteria, fungi, and some viruses. Chlorhexidine salts act as antimicrobial agents by disrupting cell membranes and causing denaturation and precipitation of the cellular content. The killing speed is dependent on solubility and concentration, with a residual effect that can last for 5-6 hours. Examples of chlorhexidine salts include but are not limited to gluconate, hydrochloride, diacetate, dimonoglycolate, succinate, diisobutyrate, dicinnamate, thiosulphate, dilactate, dicaproate, dinitrite, and diisophthalate. The most preferred salt is chlorhexidine gluconate (CHG).

Water-soluble quaternary ammonium salts used in the invention can contain a mixture of alkyl dimethyl benzyl ammonium halides with different carbon chain lengths, or alkyl pyridinium halides. The salts can function as a detergent by reducing surface tension at interfaces, while also being attracted to the negatively charged microorganism surface. Quaternary ammonium salts have primary activity against Gram positive bacteria and can have activity against Gram negative bacteria under certain conditions, such as in the presence of EDTA. Because of their high water solubility and surface active properties, they usually act in a quick manner, normally in few seconds. Examples of water soluble quaternary ammonium halides include but are not limited to benzalkonium chloride (BKC), benzethonium chloride (BZT), cetyl pyridinium chloride (CPC), dequalinium chloride, dodecyl dimethylethylbenzyl ammonium chloride, N-(3-chloroallyl)hexaminium chloride, octadecanaminium N,N,N-tris(2-hydroxylethyl) chloride, didecyl/dioctyl dimethyl ammonium chloride, dimethylethyl benzyl ammonium chloride, dimethylbenzyl ammonium chloride, and trimethoxysilyl propyloctadecyl dimethyl ammonium chloride. Quaternary ammonium halides are available under the tradename MERQUAT. An example of a preferred salt is a mixed dialkyl dimethyl ammonium chloride such as N,N-dialkyl(C₈-C₁₀)-N,N-dimethyl ammonium chloride, available as BARDAC 2050 from Lonza. Commercially available benzalkonium chloride, such as from Aldrich Chemical, Milwaukee, Wis., can contain a mixture of n-C₁₂H₂₅, n-C₁₄H₂₉, and n-C₁₆H₃₃ homologs, in various amounts such as 60-70% C₁₂, 30-40% C₁₄, and <5% C₁₆, for example. The most preferred salts are BKC, BZT and CPC.

It is not intended to link the finding of the invention to any theory, but it is believed that a miscible mixture of CHG and quaternary ammonium salts such as BKC or CPC according to the invention have a cooperative effect in killing bacteria that exist in a hospital environment in both an immediate and an extended fashion. When the treated glove surface is wetted, ammonium salts like BKC or CPC are activated instantly, and CHG activity is promoted by dissolved BKC or CPC in the aqueous environment. The antimicrobial agents, when in contact with the cell surface, destabilize outer and inner cell walls and kill the microbes. While a preferred embodiment of the invention has at least one chlorohexidine salt and at least one quaternary ammonium salt applied to the surface of a glove, it is also contemplated that the invention includes compositions and methods wherein the antimicrobial agent is a single active ingredient rather than a composition comprising more than one active ingredient. For example, it is contemplated that the invention includes a glove treated with a composition comprising a 2% CHG/acrylic polymer coating solution, and packaged accordingly.

In water-based coatings, poor wetting causes uneven coverage and defects. When a liquid that contains microorganisms contacts such uneven zones, microorganisms will survive. Wetting agents are used for deflocculating the surface and improving interaction between the coating and surface. Chemical wetting agents, like surfactants, are classified as anionic, cationic and nonionic. The ideal wetting agent has excellent surface leveling power and good compatibility with the antimicrobial agents used in the invention. Wetting agents include but are not limited to non-ionic ethoxylated alkyl phenols such as octylphenoxy polyethoxyethanol or other non-ionic wetting agents. A nonionic polyether modified dimethylpolysiloxane such as BYK-348 from BYK Chemie, Wallingford, Conn., is a preferred wetting agent.

In applying a low-solid water-based coating to a substrate, foaming often causes poor film-quality through craters, fisheyes and pinholes, especially when the predominant coating components are cationic surface active agents. Anti-foaming agents are used according to the present invent for destabilizing foaming bubbles, thereby improving wetting and distributing the antimicrobial agents uniformly. Nonionic acetylenic diols are particularly suitable for a low viscosity formulation because they provide excellent dynamic surface tension reduction during spraying and dipping coating processes. A non-limiting example of an anti-foaming agent is an acetylenic glycol-based agent available under the Dynol trade name. Other anti-foaming agents can include but are not limited to naphthalene-based compounds and silicone-based defoamers. A preferred anti-foaming agent is the ethylene glycol acetylenic diol available under the trade name Surfynol TG of Air Product and Chemical, Inc.

In order to maintain antimicrobial efficacy, the compositions of the invention are kept relatively simple since both CHG and quaternary ammonium salts are sensitive to some additives. The coating composition may additionally comprise minor ingredients as are commonly used in the art such as any of the following, either alone or in combination: humectant or skin conditioning agent, preservative, buffer, chelating agent, anti-tackifying agent, thickener, fragrance and UV absorber.

By antimicrobial efficacy is meant the reduction of the number of microbes in a sample after being contacted with a treated glove. The phrase “quick-kill” means that the antimicrobial gloves are effective in reducing the initial number of microorganisms that come into contact with the treated glove surface by at least 90% in a matter of minutes. Fast kill rates equate to better effectiveness. The term “long-lasting” is used to mean that the antimicrobial activity is maintained for a substantially long period of time, for example as a product with shelf life of about 2 years. A time period of one minute of contact is a preferred amount of time for measuring “quick-kill” antimicrobial efficacy. One feature of the antimicrobial gloves according to the invention is to kill 90% of the initial number of microorganisms, i.e. 1 log₁₀ reduction, in one to five minutes.

A preferred embodiment of the antimicrobial solution to be applied to the surface of the glove comprises an antimicrobial mixture comprising at least one water-soluble chlorhexidine salt, preferably chlorhexidine gluconate (CHG) at about 0.01% to about 4% by weight; at least one water-soluble quaternary ammonium halide, preferably benzalkonium chloride (BKC), benzethonium chloride (BZT), and/or cetyl pyridinium chloride (CPC) at about 0.5% to about 4% by weight; an aqueous carrier with or without a water-soluble alcohol; and optionally comprises one or more of the following: a wetting agent, preferably a solvent-free polyether-modified dimethylpolysiloxane (BYK-348) at about 0.01% to about 0.5% by weight, which improves coating coverage; an anti-foaming agent, preferably a self-emulsifiable acetylenic diol such as Surfynol TG at about 0.01% to about 0.3% by weight, which reduces coating defects due to dynamic surface tension reduction; a buffer or pH adjusting agent, preferably citric acid at about 0.01-0.05% by weight; a chelating agent, such as salts of ethylenediamine tetraacetic acid, preferably disodium ethylenediamine tetraacetate, disodium EDTA at about 0.1% to about 0.5% by weight; and an anti-tackifying agent. A preferred antimicrobial formulation is about 1% to about 2% by weight solids with a pH from about 4 to about 8.

Gloves according to the invention are made of natural and synthetic elastomeric material including but not limited to natural rubber, nitrile, polychloroprene, polybutadiene, polyvinylchloride, polyurethane, polyisoprene, neoprene, 2-chloro-1,3-butadiene and 2,3-dichloro, 3-butadiene, styrene diblock and triblock copolymers, graft copolymers, or other synthetic elastomers, including blends thereof. The gloves can be a single-layer or contain more than one layer in a laminate fashion. Additionally, gloves can contain standard fillers and additives. Furthermore, gloves can be coated or powdered. A preferred embodiment of the invention is essentially free of powder and essentially free of starch. By essentially free of powder and/or starch is meant, for example, less than about 2 mg of residue per glove. A particularly preferred embodiment would have no or almost no powder or starch.

While the inventors envision the application of an antimicrobial coating composition to any desired surface of the antimicrobial object, a preferred embodiment according to the invention is prepared by applying the antimicrobial coating composition to the outer surface of a medical or an industrial glove to minimize or reduce cross-contamination as a result of multiple contacts. By outside surface is meant the portion of the glove that comes into contact with other objects such as patients, medical instruments, table tops, or counters. The antimicrobial composition of this invention can also be applied to the inside surface of a surgical glove to inhibit any significant growth of skin flora. By inside surface is meant the surface that comes into contact with the wearer's hand.

The phrase “packaging protection” means that finished antimicrobial gloves are packed in a container which has a durable moisture resistance and mechanical protection. Suitable packaging material according to the invention is water- and moisture-resistant. Such packaging includes but is not limited to barrier films, metallized films, and foil laminates. A preferred embodiment of the packaging material is a metal foil pouch. An example of a preferred packaging embodiment includes but is not limited to a foil laminate of PET (polyethylene terephthalate)/aluminum foil/LDPE (low density polyethylene) from Amcor, Abbotsford Victoria of Australia, or a nylon/aluminum foil available as IntegraFlex. Additional non-limiting embodiments include SiOx laminates from Rollprint, HDPE (high density polyethylene) films available under the tradenames Perfecseal, Aclar films, Peelfrom Plus, MD Film, and PHK431, all from Amcor, Abbotsford Victoria of Australia, and MP90 from Winpak, and SK100 from Winpak. Particularly preferred packaging materials are foil laminates available as RFE 024 from Amcor and NFE 005 from Amcor. Barrier films can include but are not limited to PVDC (polyvinylidene chloride).

Suitable desiccants according to the invention maintain lower relative humidity within the packaging material compared to the external environment. Preferred desiccants include montmorillonite clay available as DESI PAK from Sud-Chemie, Belen, N. Mex. Alternatively, the desiccant can include but is not limited to silica gel, activated alumina, zeolites, molecular sieves, or calcium oxide from Sorbent Systems. A particularly preferred desiccant is anhydrous calcium sulfate available from Drierite, Xenia, Ohio.

One embodiment of the invention is also envisioned as a system comprising an antimicrobial glove and packaging providing a water-vapor-impermeable barrier. Such a system may comprise a desiccant and/or an inert water-vapor free atmosphere such as nitrogen, helium, and/or argon. By water-vapor-impermeable barrier is meant a barrier that does not permit water vapor to equilibrate across the barrier. By water-vapor free atmosphere is meant an atmosphere in the vicinity of the glove with less than 10% by weight water vapor, preferably less than 5% by weight water vapor, more preferably less than 1% by weight water vapor, and particularly preferred no or almost no water vapor.

The preferred packaging material and desiccant system provide a water-vapor impermeable barrier to maintain a low humidity level in the vicinity of the glove. A preferred embodiment reduces the relative humidity level below the relative humidity level of the ambient conditions, preferably below about 40% relative humidity, and more preferably below about 30% relative humidity. In any event, the amount of moisture in the system comprising the glove and packaging is kept to a minimum on an absolute as well as relative scale.

According to the present invention there is provided a method of packaging gloves against warm and/or humid environments to protect antimicrobial activity during storage and transportation, comprising water- and moisture-resistant packaging, preferably comprising a metal foil pouch and desiccant. Without wishing to be bound by theory, it is believed that ambient humidity can cause a reduction in antimicrobial activity for the coated gloves due to the migration of antimicrobial agent from the outer surface to interior portions of the glove. In order for a glove to have efficacy in a quick-kill test, any antimicrobial agents must be available on the surface of the glove. The migration of antimicrobial agent away from the surface decreases the availability of the antimicrobial agent on the surface of the glove, thus reducing the quick-kill efficacy of the glove. The problem is particularly acute in gloves that are essentially free of powder or essentially free of starch. The gloves of the present invention are packaged by a process wherein the packaged glove is capable of being stored and/or transported for a period of time without significant loss of antimicrobial activity. The phrase “without significant loss of antimicrobial activity” means that the packaged gloves remain effective at killing at least one log₁₀ of the number of microbes which come into contact with the gloves.

The terms “storage” and/or “transportation” are meant to encompass periods of time and conditions which are commercially reasonable for the products being stored and/or transported. The terms “coated” and “treated” are used interchangeably. Coated or treated gloves are gloves that have been subjected to an application of the active agent to a surface of the glove. By surface of the glove is meant a part of the glove that comes into contact with another surface, such as the wearer's hand, or a patient, a medical instrument, or a tabletop. Because a glove has a certain thickness, there are “interior” portions that are not on the surface of the glove. The “interior” of the glove is distinct from the inside surface, which is that part of the glove which comes into contact with the wearer's hand. The “vicinity” of a packaged glove is the remaining space within the package.

### Materials and Testing Methods

The following materials and testing methods were developed by the inventors to evaluate and address the problems in the art with respect to antimicrobial gloves. As set forth, the materials and methods were used for making and coating antimicrobial gloves, as well as in evaluating the antimicrobial efficacy of the gloves.

Antimicrobial Glove Preparation

1. Materials:

20% Chlorohexidine Gluconate (CHG) solution, Xttrium Laboratories
Benzalkonium Chloride (BKC), Aldrich Chemical, Milwaukee, Wis.
Surfynol TG, Air Products, Allentown, Pa.
BYK-348, BYK Chemie, Wallingford, Conn.
Nitrile Glove, On-line, Syntex, China
Natural Rubber Glove, YTY, Malaysia
   2. Antimicrobial Solution Preparation:

An antimicrobial solution for coating a glove surface was made by blending a wetting solution, a BKC solution and a CHG solution followed by continuous stirring until a clear solution was formed.

For example, a 500 g wetting solution, containing 2% Surfynol TG and 1% BYK 348, was made by adding Surfynol TG (10 g) and BYK-348 (5 g) into deionized water (485 g). A 50% BKC solution was made by mixing 51.65 g of BKC with 103.3 mL of deionized water and stirring the solution for 1 h. A 1.9% CHG solution was made by mixing 96.8 g CHG (20% solution) and 1 L deionized water. A 1.25% antimicrobial solution was made by diluting 4.57 g wetting solution made above with 6 lb deionized water in a clean tank, adding 76.2 g of the 50% BKC solution made above into the tank and finally adding 1096.8 g of the CHG solution made above into the tank and stirring the solution.

3. Glove Surface Coating Treatment:

A glove surface was treated by a dipping process. A glove was placed on a former. The former was inverted and dipped in the antimicrobial solution prepared above for 10 seconds. While the former was still inverted, the dipping tank was removed and the glove was allowed to drip dry for 10 seconds. The glove was placed in an oven for 20 minutes at 70° C. The glove was removed from the oven and allowed to cool to room temperature (approximately 20 minutes). The glove was removed from the former.

A glove surface was also treated by a spraying-process. A glove was placed on a former. An antimicrobial solution was poured into an atomizer. The glove was sprayed twice on each side of the former. The glove was placed in an oven for 20 minutes at 70° C. The glove was removed from the oven and allowed to cool to room temperature (approximately 20 minutes). The glove was removed from the former.

Antimicrobial Agents Loading Level and Ratio of BKC/CHG:

The loading level of antimicrobial agents coated on the glove surface was controlled by the type of antimicrobial agents, the total solid content of the antimicrobial coating composition, the application process, e.g. dipping or spraying, the treatment conditions, drying temperature, and time. For example, 1.5% means 100 parts of weight of an antimicrobial coating composition having 1.5 parts by weight of a solid antimicrobial agent. The relative amount of BKC and CHG in the antimicrobial coating composition was measured by the weight ratio of the two ingredients. For example, BKC/CHG=2/1 means that the ratio of BKC was twice the amount by weight of CHG.

Antimicrobial Testing Methods

In order to evaluate the antimicrobial efficacy of the gloves, tests were developed in order to make the required comparisons. Once the loading level of the antimicrobial agent on the gloves was determined, the effectiveness of the antimicrobial gloves was measured by the log reduction in a “Time-Kill” test.

1. Materials—ATCC (American Type Culture Collection):

Pseudomonas aeruginosa, ATCC # 15442; Escherichia coli, ATCC # 11229; Staphylococcus aureus, ATCC # 6538; Enterococcus faecalis, ATCC # 29212; Enterobacter cloacae, ATCC # 13047; Staphylococcus epidermidis, ATCC # 12228; Candida albicans, ATCC # 10231. Source: Manufactured by MicroBioLogics, Inc. Saint Cloud, Minn. 56303 (Distributed by Biomerieux, Ind.) Lyophilized microorganisms (lab stock cultures). Clinical Isolates—Laboratory Stock Cultures: Enterococcus faecalis, VRE; Staphylococcus aureus, MRSA. Source: Microbiology Laboratories of Victory Memorial Hospital, Waukegan, Ill.

2. Challenge Microbial Suspension

Well-isolated 24-hour growth colonies of the same morphological type from an agar plate were transferred in 4-5 mL of sterile saline in order to prepare microbial suspensions that have turbidity matches to McFarland Turbidity Standard No. 0.5.

3. Inoculum Titer

Twenty microliters of the challenge suspension were mixed well with 10 mL of neutralizing solution. Ten-fold dilutions from 10⁻¹ to 10⁻³ were made by transferring 0.22 mL into 2 mL neutralizing solution. The organisms were inoculated onto agar plates by traditional bacterial techniques with duplicated 0.2 mL inocula and incubated under conditions appropriate for the individual microorganism for 24 hours. After incubation, the growth colonies on the plates were manually counted, and the inoculum titer was calculated. The final concentration of the inoculum titer was about 1.5×10⁵ CFU/ml.

Effectiveness of Antimicrobial Gloves (Log Reduction)

Testing glove samples were aseptically cut from the palm areas to approximately 1 square inch. The outside surface of the cut gloves was identified. A small quantity of bacterial culture, e.g. 10 or 20 microliters of the challenge microbial suspension, was added onto a sterile glass coverslip (18 mm×18 mm), which was placed in contact with a cut coated glove surface for a designated time interval, such as 1 and/or 5 minutes, at room temperature. At the end of the time exposure, both the glove material and the coverslip were dropped into a test tube containing 10 mL of neutralizing agent. Ten-fold dilutions from 10⁻¹ to 10⁻³ were made by transferring 0.22 mL into 2 mL neutralizing solution. One ml from the 10 ml neutralization solution containing the glove material and the coverslip, and 0.2 ml in duplicate from the rest of the dilutions were enumerated for surviving bacteria using standard agar plating methods. Results were reported on a logarithmic scale.

Aging of Packaged and Unpackaged Gloves

The temperature and relative humidity at the glove surface were controlled for a specific period of time in order to simulate potential storage or transportation conditions. The variables involved in the packaging procedure included the number of gloves, the nature of the packaging material (desiccant and barrier/laminate), the packaging configuration, and the processing condition (seal temperature and seal time). A typical package for sale will contain 100 gloves. An antimicrobial solution with total solids content from about 1-5% by weight was applied to the glove surface to be tested. Gloves to be tested included nitrile and natural rubber gloves. Possible packaging combinations included a Nylon/Aluminum Foil/LDPE (NFE) pouch from Amcor, Abbotsford Victoria of Australia, and a calcium sulfate desiccant bag (2.5 g) made by Drierite, Xenia, Ohio, or a PET/Aluminum Foil/LDPE (RFE) pouch from Amcor, Abbotsford Victoria of Australia and a clay/DesiPak made by Sud-Chemie, Belen, N. Mex.

The package containing from two to twenty gloves and desiccant was sealed at 200° C. for 2.0 seconds and cooled at 85° C. using a Pack World Sealer #30. The package was placed in a chamber where it was exposed to 70% humidity and 40° C. for the specified time.

### EXAMPLES

In order that the present invention may be more readily understood, specific non-limiting examples are shown below.

### Example 1

### Broad Spectrum Antimicrobial Activity of Treated Gloves

Gloves were treated by dipping the gloves into an antimicrobial coating composition of CHG/BKC prepared as described above. Nitrile gloves from Syntex, China, were used. The concentration of the antimicrobial coating was 1.5% by weight and the ratio of BKC/CHG was 2/1. The gloves were dried at 60° C. for 30 minutes and tested in a one-minute test.

TABLE 1
Antimicrobial effectiveness of BKC/CHG treated gloves against
various microorganisms

log reduction

| Microorganisms | uncoated | coated |
|---|---|---|
| Staphylococcus aureus | 0.87 | 4.42 |
| Escherichia coli | 0.28 | 5.41 |
| Pseudomonas aeruginosa | 0.22 | 3.00 |
| Enterococcus faecalis | 0.13 | 3.67 |
| MRSA | 0.09 | 2.51 |
| VRE | 0.12 | 3.12 |
| Candida albicans | 0.05 | 2.90 |

The data in Table 1 show the antimicrobial activity of CHG/BKC coated gloves against a broad spectrum of microorganisms. Larger values for the log reduction indicate greater antimicrobial efficacy in the “Time-Kill” test.

### Example 2

### Broad Spectrum Antimicrobial Activity of Surgical Gloves

The data in Table 2 below illustrate that sterilized surgical gloves also have broad spectrum activity. Polyisoprene surgical gloves were coated on the inside surface by a CHG/CPC coating solution and were sterilized by a Gamma irradiation process. The log₁₀ reduction was tested for glove antimicrobial activity before and after sterilization.

Glove preparation: A 40 lb solution of 1.55% CPC and 0.5% CHG was made by adding 281.3 g of CPC, 453.6 g of CHG, 25.7 g of wetting agent (2% Surfynol TG and 1% BYK 348) and 39.5 lbs of deionized water. The wetting agent was prepared by weighing 0.514 g of Surfynol TG and 0.257 g of BYK 348 into a 100 ml beaker. Deionized water (26 ml) was added and the solution stirred for 30 min. The antimicrobial solution was used to dip 180 gloves. The solution was changed every 40 gloves. The surgical glove was placed on a former, dipped in the tank for 10 sec, and dripped dry for 10 sec. The gloves were placed in an oven for 60 min at 45° C. The gloves were packaged in wallets and sleeves to be sealed for sterilization. The gloves were sterilized using Gamma irradiation at a dosage of 38.5-39 KGY. The activity was measured for the final gloves and the results are summarized in table below.

TABLE 2
Antimicrobial effectiveness of sterilized gloves against various
microorganisms

| | |
|---|---|
| Microorganisms/ | Log reduction |

| contact time | Before sterilization | After sterilization |
|---|---|---|
| Staphylococcus | | |
| aureus | | |
| 1 min | >4.77 | >5.32 |
| 5 min | | >5.76 |
| Staphylococcus | | |
| epidermidis | | |
| 1 min | >3.77 | 4.04 |
| 5 min | | >5.60 |
| Enterococcus | | |
| faecalis | | |
| 1 min | >4.28 | >5.45 |
| 5 min | | >5.45 |
| VRE | | |
| 1 min | >3.88 | 4.00 |
| 5 min | | 5.42 |
| MRSA | | |
| 1 min | 3.17 | 4.29 |
| 5 min | | >5.97 |

The results shown in Table 2 indicate that sterilized polyisoprene surgical gloves treated on the inside surface by an antimicrobial coating solution containing 0.5% CHG and 1.55% CPC provided excellent antimicrobial activities against a broad spectrum of microbes.

### Example 3

### Effect of Packaging on Storage Stability for Natural Rubber Gloves

The storage stability of gloves treated with a water-based coating according to the invention was tested by measuring antimicrobial activities as described above following the aging process as described above.

In Table 3A, the total solids content of the antimicrobial solution was 3%, the ratio of BKC/CHG was 2/1, and 2-3 gloves were packaged in a Nylon/Aluminum Foil/LDPE (NFE) pouch from Amcor, Abbotsford Victoria of Australia, and a calcium sulfate desiccant bag (2.5 g) made by Drierite, Xenia, Ohio.

TABLE 3A
Effect of packaging on antimicrobial activity (log reduction) for
treated natural rubber gloves (YTY, Malaysia), 3% total solids in
antimicrobial coating compositions

| micro- | | Aged/Not packaged | | | Aged/Packaged | | | |
|---|---|---|---|---|---|---|---|---|
| organisms/ | Not | (days) | | | (days) | | | |
| contact time | aged | 3 | 10 | 20 | 3 | 10 | 20 | 45 |
| Staphylococcus | | | | | | | | |
| aureus | | | | | | | | |
| 1 minute | 4.04 | 3.53 | 2.45 | 1.08 | 4.93 | 4.18 | 4.17 | 3.50 |
| 5 minutes | 4.76 | 4.52 | 3.64 | 1.81 | 5.44 | 4.60 | >5.66 | 3.80 |
| Pseudomonas | | | | | | | | |
| aeruginosa | | | | | | | | |
| 1 minute | 4.08 | 0.35 | 0.03 | 0.09 | 4.18 | 2.52 | 2.94 | 2.90 |
| 5 minutes | 4.94 | 1.35 | 0.26 | 0.36 | 4.25 | 4.52 | 4.14 | 3.23 |

Data show that with the packaging, the natural rubber gloves maintain a significant amount of their antimicrobial activity up to and after 45 days while the gloves without packaging lost their activity after only three days of aging. For example, the one minute result for Staphylococcus aureus after 45 days of aging for the packaged gloves is within one log unit of the result for gloves that were not aged.

In Table 3B, total solids contents of the antimicrobial solution was 1.25%. The ratio of BKC/CHG was 2/1, and 2-3 gloves were packaged in PET/Aluminum Foil/LDPE (RFE) of Amcor, Abbotsford Victoria of Australia and a clay/Desi Pak from Sud-Chemie, Belen, N. Mex.

TABLE 3B
Effect of packaging on antimicrobial activity (log reduction) for treated
natural rubber gloves (YTY, Malaysia), 1.25% total solids
(BKC/CHG = 2/1, 70° C. dry)

| | | Aged/Packaged | | |
|---|---|---|---|---|
| microorganisms/ | Not | (days) | | |
| contact time | aged | 3 | 10 | 30 |
| Staphylococcus | | | | |
| aureus | | | | |
| 1 minute | >5.20 | 5.10 | 5.11 | 4.90 |
| 5 minutes | >5.20 | 5.26 | 4.25 | 4.90 |
| Pseudomonas | | | | |
| aeruginosa | | | | |
| 1 minute | 4.82 | 4.51 | 2.72 | 4.00 |
| 5 minutes | >5.12 | 5.40 | 3.30 | 4.00 |

Table 3B shows that the log reduction result in the one minute Staphylococcus aureus test after 30 days of aging for gloves packaged according to the invention remains within one log of the result for unaged gloves. The tests for unpackaged gloves were not performed, as the data in Table 3A is sufficient to show that unpackaged gloves quickly lose their antimicrobial efficacy.

In Table 3C, total solids contents of the antimicrobial solution was 0.75%. The ratio of BKC/CHG was 2/1, and 2-3 gloves were packaged in PET/Aluminum Foil/LDPE (RFE) of Amcor, Abbotsford Victoria of Australia and a clay/Desi Pak from Sud-Chemie, Belen, N. Mex.

TABLE 3C
Effect of packaging on antimicrobial activity (log reduction) for treated
natural rubber gloves (YTY, Malaysia), 0.75% total solids
(BKC/CHG = 2/1, 70° C. dry)

| | | |
|---|---|---|
| | | Aged/Packaged |
| microorganisms/ | Not | (days) |

| contact time | aged | 3 | 30 |
|---|---|---|---|
| Staphylococcus | | | |
| aureus | | | |
| 1 minute | 4.50 | 5.10 | 4.50 |
| 5 minutes | 4.84 | 5.26 | 4.90 |
| Pseudomonas | | | |
| aeruginosa | | | |
| 1 minute | 4.19 | 2.26 | 2.25 |
| 5 minutes | 5.12 | 3.43 | 4.00 |

Table 3C shows that the log reduction result in the one minute Staphylococcus aureus test after 30 days of aging for gloves packaged according to the invention remains about the same as the log reduction result for unaged gloves. The tests for unpackaged gloves were not performed, as the data in Table 3A is sufficient to show that unpackaged gloves quickly lose their antimicrobial efficacy.

### Example 4

### Effect of Packaging on Storage Stability for Nitrile Rubber Gloves

In Table 4A, the total solids content of the antimicrobial solution was 3%, the ratio of BKC/CHG was 2/1, and 2-3 gloves were packaged in a Nylon/Aluminum Foil/LDPE (NFE) pouch from Amcor, Abbotsford Victoria of Australia, and a calcium sulfate desiccant bag (2.5 g) made by Drierite, Xenia, Ohio.

TABLE 4A
Effect of packaging on antimicrobial activity (log reduction) for treated
nitrile rubber gloves (Syntex, China)

| | | Aged/Not packaged | | | | | Aged/Packaged | | | | |
|---|---|---|---|---|---|---|---|---|---|---|---|
| microorganisms/ | Not | (days) | | | | | (days) | | | | |
| contact time | aged | 3 | 6 | 10 | 25 | 45 | 3 | 6 | 10 | 25 | 45 |
| Staphylococcus | | | | | | | | | | | |
| aureus | | | | | | | | | | | |
| 1 minute | 5.78 | 1.18 | 0.06 | 0.17 | 0.04 | 0.11 | 5.00 | 5.52 | 4.76 | 5.00 | 4.75 |
| 5 minutes | 5.78 | 1.74 | 0.72 | 0.61 | 0.75 | 0.31 | 5.07 | 4.88 | 4.93 | 4.73 | 5.21 |
| Pseudomonas | | | | | | | | | | | |
| aeruginosa | | | | | | | | | | | |
| 1 minute | 5.87 | 0.35 | 0.43 | 0.08 | 0.06 | 0.08 | >5.97 | >5.97 | 5.77 | 4.07 | 4.07 |
| 5 minutes | 5.87 | 1.06 | 0.49 | 0.43 | 0.02 | 0.35 | 4.61 | >5.97 | >5.87 | 4.66 | 5.64 |

Data show that with the packaging according to the invention, the nitrile rubber gloves maintain their antimicrobial activities within one log of their original activity in the five minute Staphylococcus aureus test after 45 days while the gloves without packaging lost most of their activity after only three days of aging.

In Table 4B, total solids contents of the antimicrobial solution was 1.5%. The ratio of BKC/CHG was 2/1, and 2-3 gloves were packaged in PET/Aluminum Foil/LDPE (RFE) of Amcor, Abbotsford Victoria of Australia and a clay/Desi Pak from Sud-Chemie, Belen, N. Mex.

TABLE 4B
Effect of packaging on antimicrobial activity (log₁₀ reduction)
for treated nitrile rubber gloves (Syntex, China), 1.5% total solids
(BKC/CHG = 2/1, 70° C. dry)

| | |
|---|---|
| | Aged/Packaged |
| microorganisms/ | (days) |

| contact time | Not aged | 3 | 45 |
|---|---|---|---|
| Staphylococcus | | | |
| aureus | | | |
| 1 minute | 5.33 | 3.62 | 4.04 |
| 5 minutes | 5.33 | 5.21 | 5.16 |
| Pseudomonas | | | |
| aeruginosa | | | |
| 1 minute | 5.06 | 5.27 | 1.52 |
| 5 minutes | 4.00 | 5.88 | 3.97 |

The data in Table 4B show that with the packaging according to the invention, the nitrile rubber gloves maintain their antimicrobial activities within one log of their original activity in the five minute Staphylococcus aureus and Pseudomonas aeruginosa tests after 45 days.

In Table 4C, total solids contents of the antimicrobial solution was 1.25%. The ratio of BKC/CHG was 2/1, and 2-3 gloves were packaged in PET/Aluminum Foil/LDPE (RFE) of Amcor, Abbotsford Victoria of Australia and a clay/Desi Pak from Sud-Chemie, Belen, N. Mex.

TABLE 4C
Effect of packaging on antimicrobial activity (log₁₀ reduction) for
treated nitrile rubber gloves (Syntex, China), 1.25% total solids
(BKC/CHG = 2/1, 70° C. dry)

| | | Aged/Packaged |
|---|---|---|
| microorganisms/ | Not | (days) |
| contact time | aged | 45 |
| Staphylococcus | | |
| aureus | | |
| 1 minute | 5.20 | 3.33 |
| 5 minutes | 5.20 | 5.18 |
| Pseudomonas | | |
| aeruginosa | | |
| 1 minute | 4.92 | 3.16 |
| 5 minutes | 5.12 | 4.87 |

The data in Table 4C show that with the packaging according to the invention, the nitrile rubber gloves maintain their antimicrobial activities within one log of their original activity in the five minute Staphylococcus aureus and Pseudomonas aeruginosa tests after 45 days even at reduced concentrations of antimicrobial agents (1.25% as compared to 3% and 1.5% in the previous tests).

In Table 4D, total solids contents of the antimicrobial solution was 1.5%. The ratio of Bardac/CHG was 2/1, and 2-3 gloves were packaged in PET/Aluminum Foil/LDPE (RFE) of Amcor, Abbotsford Victoria of Australia and a clay/Desi Pak from Sud-Chemie, Belen, N. Mex.

TABLE 4D
Effect of packaging on antimicrobial activity (log₁₀ reduction)
for treated nitrile rubber gloves (Syntex, China), 1.5% total solids
(Bardac/CHG = 2/1, 70° C. dry)

| | | |
|---|---|---|
| | | Aged/Packaged |
| microorganisms/ | Not | (days) |

| contact time | aged | 3 | 45 |
|---|---|---|---|
| Staphylococcus | | | |
| aureus | | | |
| 1 minute | 4.27 | 2.88 | 2.52 |
| 5 minutes | 4.79 | 5.51 | 3.73 |
| Pseudomonas | | | |
| aeruginosa | | | |
| 1 minute | 4.52 | 4.79 | 1.82 |
| 5 minutes | 4.38 | 5.50 | 2.62 |

The data in Table 4D show that with the packaging according to the invention, the nitrile rubber gloves maintain their antimicrobial activities within approximately one log of their original activity in the five minute Staphylococcus aureus test after 45 days. The data for the unpackaged gloves was not continued, since unpackaged gloves were shown to lose activity in Table 3A. The data in Table 4D with Bardac instead of BKC show that the protection accorded to the antimicrobial gloves by the packaging according to the present invention is a general phenomenon rather than specific for gloves comprising BKC.

### Example 5

### Effect of Aging on Antimicrobial Activity for Various Coated Substrates

A 1.5% antimicrobial coating solution containing 1% BKC and 0.5% CHG was prepared as described previously. SP Microslides were placed in a Petri dish and 0.05 ml of 1% BKC/0.5% CHG were added on to the frosted side of the slide. The slides were placed in an oven and dried for 60 min at 45° C. Nitrile gloves (Syntex, China, Lot# 6311A) were treated with the 1.5% antimicrobial solution as described previously. Both slides and treated gloves were placed in an aging oven at 40° C. and 75% relative humidity. Antimicrobial activity against Staphylococcus aureus and Pseudomonas aeruginosa was tested as described previously at 0, 3 or 4, and 10 days aging.

TABLE 5
Aging effect on antimicrobial activities (log₁₀ reduction) for coated
glass surface vs. coated nitrile rubber gloves (Syntex, China)

| | | |
|---|---|---|
| | Glass surface | Glove surface |
| Microorganisms/ | (days aged) | (days aged) |

| contact time | 0 | 4 | 10 | 0 | 3 |
|---|---|---|---|---|---|
| Staphylococcus | | | | | |
| aureus | | | | | |
| 1 min | >3.24 | >3.00 | >3.85 | 5.38 | 0.18 |
| 5 min | >3.78 | >3.68 | >3.88 | 5.68 | 1.04 |
| Pseudomonas | | | | | |
| aeruginosa | | | | | |
| 1 min | 2.43 | 1.67 | 1.55 | 3.81 | 0.07 |
| 5 min | >3.68 | 2.39 | 2.81 | 5.18 | 0.10 |

This example illustrates that the nature of the substrate treated by the antimicrobial coating composition of the invention affected long-term antimicrobial activity. The treated surfaces were aged under increased temperature and humidity conditions. The two substrates investigated were glass surface and medical glove surface. The results unexpectedly showed that cured rubber surfaces such as the glove surface used in the study and glass surface responded differently to the aging process. For the treated glove surface, antimicrobial activity was almost completely lost after three days of aging, while the treated glass surface maintained its antimicrobial activity after at least 10 days of aging. While not wishing to be bound by theory, it is thought that the aging process facilitated migration of the antimicrobial agents CHG and BKC into the interior of the glove, such that the antimicrobial agents were no longer available on the surface, with a resulting loss in antimicrobial activity.

As shown in the tables, the packaging structure protects antimicrobial-treated natural rubber gloves and nitrile rubber gloves from moisture attack and maintains significant antimicrobial activity after several days of aging. Without wishing to be bound by theory, it is believed that the gloves are protected from moisture that accelerates the migration of water-soluble CHG and BKC to the interior of the gloves by the packaging system, and as a result, can maintain antimicrobial efficacy even at relatively low concentrations (i.e. 0.75% in the case of natural rubber gloves) of CHG and BKC. Again, without wishing to be bound by theory, the differences between the results for natural rubber and nitrile rubber gloves are attributed to differences in migration rates into the underlying substrate.

From the above description, one can ascertain the essential characteristics of the present invention and, without departing from the spirit and scope thereof, can make various changes and modifications of the invention to adapt it to various uses and conditions.

1. A packaged antimicrobial elastomeric article comprising an elastomeric article treated on the surface with at least one antimicrobial agent, and a package, and a means for reducing the relative humidity in the vicinity of the elastomeric article to less than the ambient relative humidity.

2. The packaged article of claim 1, wherein the article is a glove, and further wherein the at least one antimicrobial agent comprises chlorhexidine gluconate and at least one quaternary ammonium halide selected from benzalkonium chloride and/or cetyl pyridinium chloride.

3. The package of claim 2, wherein the means for reducing the relative humidity in the vicinity of the glove is a desiccant and a sealed moisture-resistant barrier or metal foil pouch containing the desiccant.

4. The package of claim 2, wherein the relative humidity in the vicinity of the glove is less than about 40% relative humidity.

5. The package of claim 2, wherein the surface of the glove is essentially free of powder.

6. The package of claim 2, wherein the surface of the glove is essentially free of starch.

7. A method of producing an antimicrobial elastomeric article comprising:
forming an elastomeric article, applying at least one antimicrobial agent to the surface of the elastomeric article, and packaging the elastomeric article in a means for reducing the relative humidity in the vicinity of the elastomeric article to less than the ambient relative humidity.

8. The method of claim 7, wherein the elastomeric article is a glove.

9. The method of claim 8, wherein the at least one antimicrobial agent comprises at least one water-soluble chlorhexidine salt and at least one water-soluble quaternary ammonium halide.

10. The method of claim 9, wherein the water-soluble chlorhexidine salt is chlorhexidine gluconate.

11. The method of claim 9, wherein the water-soluble quaternary ammonium halide is benzalkonium chloride and/or cetyl pyridinium chloride.

12. The method of claim 10, wherein the antimicrobial agent is applied to the surface of the glove by spraying or dipping.

13. The method of claim 8, comprising packaging the glove with a means for reducing the relative humidity in the vicinity of the glove to less than about 30% relative humidity.

14. The method of claim 13, wherein the means for reducing the relative humidity in the vicinity of the glove to less than about 30% is a moisture-resistant barrier or metal foil pouch with a desiccant.

15. The method of claim 12, wherein the antimicrobial agent is applied to both the outside surface and the inside surface of the glove.

16. The method of claim 12, wherein the antimicrobial agent is applied to the outside surface of the glove.

17. The method of claim 12, wherein the antimicrobial agent is applied to the inside surface of the glove.

18. The method of claim 8, wherein the antimicrobial activity of the glove after storage for 45 days exhibits at least 1 log₁₀ reduction of the initial number of microorganisms that come into contact with the treated glove surface in one minute of contact.

19. The method of claim 8, wherein the gloves are essentially free of powder.

20. The method of claim 8, wherein the gloves are essentially free of starch.

21. A method of preserving and/or prolonging the antimicrobial efficacy of an elastomeric article, said method comprising:
obtaining an elastomeric article;
applying at least one antimicrobial agent to a surface of the elastomeric article;
and packaging the elastomeric article with a means for reducing the relative humidity in the vicinity of the elastomeric article within the package to less than the ambient relative humidity,
wherein by antimicrobial efficacy is meant at least 1 log₁₀ reduction in the initial number of microorganisms in a sample that come into contact with the treated elastomeric article surface due to one minute of contact with said elastomeric article.

22. The method according to claim 21, wherein the elastomeric article is a glove.

23. The method according to claim 22, wherein the at least one antimicrobial agent comprises at least one chlorhexidine salt and at least one quaternary ammonium halide.

24. The method according to claim 22, wherein the glove is essentially free of starch and/or powder.

25. The method according to claim 22, comprising packaging the glove with a means for reducing the relative humidity in the vicinity of the glove to less than about 30% relative humidity.

26. The method according to claim 22, wherein the microorganisms comprise Staphylococcus aureus and/or Pseudomonas aeruginosa.

27. An antimicrobial elastomeric article comprising an elastomeric article coated with at least one antimicrobial agent wherein the antimicrobial elastomeric article is packaged by a process that extends the antimicrobial activity of the elastomeric article compared to an unpackaged elastomeric article comprising:
forming an elastomeric article, applying at least one chlorhexidine salt and at least one quaternary ammonium halide to a surface of the elastomeric article, and packaging the elastomeric article in a means for reducing the relative humidity in the vicinity of the elastomeric article to less than the ambient relative humidity,
wherein the packaged elastomeric article is capable of being stored and/or transported for a period of time without significant loss of antimicrobial activity.

28. The elastomeric article of claim 27, wherein the elastomeric article is a glove.

29. The glove of claim 28, wherein the at least one antimicrobial agent comprises at least one chlorhexidine salt and at least one quaternary ammonium halide.

30. The glove of claim 28, wherein the period of time is at least 45 days.

31. The glove of claim 28, wherein the antimicrobial activity of the glove after storage for 45 days exhibits at least 1 log₁₀ reduction of the initial number of microorganisms that come into contact with the treated glove surface in one minute of contact.

32. The glove of claim 28, wherein the glove is essentially free of starch and powder.

33. The glove of claim 28, wherein the means for reducing the relative humidity in the vicinity of the glove is a moisture-resistant barrier container or metal foil pouch with a desiccant.

34. The glove of claim 29, wherein at least one chlorhexidine salt is chlorhexidine gluconate and at least one quaternary ammonium halide is benzalkonium chloride and/or cetyl pyridinium chloride.

35. An antimicrobial glove and packaging system comprising an elastomeric glove, an antimicrobial agent, and a water-vapor-impermeable package.

36. The system of claim 35, wherein the antimicrobial agent comprises at least one chlorhexidine salt and at least one quaternary ammonium halide.

37. The system of claim 36, wherein at least one chlorhexidine salt is chlorhexidine gluconate and at least one quaternary ammonium halide is benzalkonium chloride and/or cetyl pyridinium chloride.

38. The system of claim 35, wherein the water-vapor-impermeable package comprises a moisture-resistant barrier container or metal foil pouch with a desiccant and/or an inert water-vapor free atmosphere.

39. The system of claim 38, wherein the atmosphere in the water-vapor-impermeable package contains less than 10% water vapor by weight.

40. The system of claim 39, wherein the atmosphere in the water-vapor-impermeable package contains less than 5% water vapor by weight.

41. The system of claim 40, wherein the atmosphere in the water-vapor-impermeable package contains less than 1% water vapor by weight.

42. The system of claim 35, wherein the glove is essentially free of starch and/or powder.
